(19) United States
(12) Patent Application Publication
(10) Pub. No.: US 2026/0069875 A1
(43) Pub. Date: Mar. 12, 2026

(54) ROTATING ELECTRIC FIELD APPARATUS AND METHOD FOR ELIMINATING HIGH FREQUENCY ARRHYTHMIC ELECTRICAL STATES OF BIOLOGICAL ORGANS AND TISSUES

(71) Applicant: Georgia Tech Research Corporation, Atlanta, GA (US)

(72) Inventors: Ilija Uzelac, Atlanta, GA (US); Flavio Fenton, Atlanta, GA (US)

(21) Appl. No.: 19/106,418

(22) PCT Filed: Aug. 30, 2023
(86) PCT No.: PCT/US2023/073148

§ 371 Date: Feb. 25, 2025

Related U.S. Application Data
(60) Provisional application No. 63/374,031, filed on Aug. 31, 2022.

(51) Int. Cl. A61N 1/39

(52) U.S. Cl. CPC A61N 1/3918; A61N 1/395; A61N 1/3987

(57) ABSTRACT

A method of reducing arrhythmic electrical states in a biological tissue, the method comprising: placing a plurality of electrode pairs around the biological tissue; and sequentially applying an electric charge to the plurality of electrode pairs. Each electric charge applied to the plurality of electrodes pairs can stimulate a distinct portion of the biological tissue. Placing the plurality of electrode pairs around the biological tissue can comprise: placing a first electrode pair around a first portion of the biological tissue; and placing a second electrode pair around a second portion of the biological tissue.

### CROSS-REFERENCE TO RELATED APPLICATIONS

This application claims the benefit of U.S. Provisional Application Ser. No. 63/374,031, filed on 31 Aug. 2022, which is incorporated herein by reference in its entirety as if fully set forth below.

### GOVERNMENT LICENSE RIGHTS

This invention was made with government support under Agreement Nos. R01HL1434050 and R15HL147348, awarded by National Institutes of Health, and Agreement No. 1446675, awarded by National Science Foundation. The government has certain rights in the invention.

### FIELD OF THE DISCLOSURE

The various embodiments of the present disclosure relate generally to systems and methods for reducing arrhythmic electrical states of biological organs and tissues.

### BACKGROUND

Cardiovascular disease is the main cause of death in developed countries, and over the last five years, it has been the leading cause in the US, with almost as many as the next two causes (cancer and stroke) combined. From all deaths attributed to heart disease, sudden cardiac arrest (SCA) represents approximately half. The specific causes of SCA can vary; however, most commonly it results from hemodynamic collapse primarily attributed to cardiac arrhythmias, such as ventricular fibrillation (VF) or ventricular tachycardia (VT) degenerating into VF. Other prevalent arrhythmic diseases include atrial fibrillation (AF), which is the most common arrhythmia worldwide, affecting 33.5 million people. Complications associated with chronic AF include an increased risk for both thromboembolism and stroke secondary to clot formation in the fibrillating atria. Accordingly, AF is the most important cause of ischemic stroke in the elderly.

For VF, drugs such as amiodarone can be used for prevention; however, the only effective treatment is a high-energy defibrillation shock, and the sooner it is applied, the higher the survival rate. Nevertheless, even the several Joules per shock for an implantable cardioverter defibrillator (ICD) can result in adverse effects like severe pain and anxiety disorders from mis-firings along with myocardial damage from repeated shocks, which can lead to increased mortality. VT treatments include ICD implantation, anti-arrhythmic drug (AAD) therapy and catheter ablation (CA). AADs can reduce VT recurrence, but can have significant limitations in VT treatment and side effects/toxicity as well as limited long-term efficacy before drug intolerance. CA can stop and prevent VT, but its effectiveness depends on the underlying disease, risks are associated, and there remains limited evidence to show overall survival improvement compared to ICDs. ICDs, however, do not prevent ventricular arrhythmias, and although frequently lifesaving, they can negatively affect quality of life in patients who experience recurrent inappropriate shocks.

AF is notoriously difficult to treat. Standard electrical cardioversion has significant side effects including electroporation, tissue damage, pain, and sedation risks; chemical cardioversion also can have side effects and has levels of success ranging from <27% to >85% depending on the drug, delivery method (oral vs. intravenous), dosage, and patient health, among others. Radiofrequency ablation (RFA) has a success rate of >60% for paroxysmal AF, but <30% success for persistent AF. Given these low success rates, the prevalence of the disease and the fact that pharmacological and non-pharmacological approaches to AF management have not been uniformly successful, it is evident that new approaches are needed to treat AF.

Clinical experience suggests that AF is a self-promoting electrical disease. Paroxysmal AF often progresses to permanent AF, and restoration and maintenance of sinus rhythm becomes more difficult as the duration of AF preceding conversion increases. Although underlying anatomic or pathophysiologie factors may fuel the progression from paroxysmal to permanent AF, compelling data suggest that AF itself may facilitate its own progression and perpetuation. The finding that AF begets AF through the remodeling of not only the electrical but also the structural and metabolic properties of atrial cells has critically changed the approach to AF treatment. It has led to management strategies such as early cardioversion and treatments employing atrial defibrillators and anti-tachycardia pacing (ATP). These treatments are intended to avoid the progression of paroxysmal and persistent AF to permanent AF by reducing the frequency and duration of AF episodes.

There are many problems with current AF/VT/VF treatment approaches. Cardioversion and defibrillation attempt to reset all the heart's electrical activity, which requires strong shocks (130-360 J and up to 720 J external, 7 J internal), eliciting large (>5 V/cm) electrical field gradients everywhere in the tissue. It is impossible to satisfy this condition without some areas having voltage gradients above the electroporation threshold, which can be as low as 10 V/cm at tissue heterogeneities like blood-muscle interfaces. Consequently, the high electrical field strengths required by conventional methods can cause myocardial damage, considerable pain for the conscious patient, and, when delivered by an implanted device, shorten battery life with each shock. These shortcomings have led to alternative electrical therapies. One alternative is to target the locations of focal stimuli implicated in triggering AF and sometimes VT/VF. For AF, catheter-based techniques have been developed that electrically isolate the pulmonary veins, the most common nidus of AF, from the remainder of the atria, as well as by targeting rotors. Although ablation is effective in most patients with paroxysmal AF, it still fails to cure 20-25% of them and more than 70% of patients with persistent AF. It also increases the risk of serious complications, including cardiac tamponade, phrenic nerve paralysis and atrioesophageal fistulae. Latent pacemakers may be suppressed using ATP; however, ATP is not generally effective in converting established AF. Accordingly, it is clear that there is a need for more effective approaches for the treatment of AF, VT and VF.

### BRIEF SUMMARY

An exemplary embodiment of the present disclosure provides a method of reducing and/or eliminating arrhythmic electrical states in a biological tissue. The method can comprise: placing a plurality of electrode pairs around the biological tissue; and sequentially applying an electric charge to of the plurality of electrode pairs.

In any of the embodiments disclosed herein, each electric charge applied to the plurality of electrodes pairs can stimulate a distinct portion of the biological tissue.

In any of the embodiments disclosed herein, placing the plurality of electrode pairs around the biological tissue can comprise: placing a first electrode pair around a first portion of the biological tissue; and placing a second electrode pair around a second portion of the biological tissue.

In any of the embodiments disclosed herein, sequentially applying an electric field can comprise: applying a first electric charge across the first pair of electrodes; and, after applying the first electric charge across the first pair of electrodes, applying a second electric charge across the second pair of electrodes.

In any of the embodiments disclosed herein, sequentially applying the electric charge to each of the plurality of electrodes can result in a rotating electric field across the biological tissue.

In any of the embodiments disclosed herein, sequentially applying the electric charge to each of the plurality of electrodes can result in a three-dimensional rotating electric field across the biological tissue.

In any of the embodiments disclosed herein, the electric charges applied to the plurality of electrodes can be electric charge pulses.

In any of the embodiments disclosed herein, each electric charge pulse can have a duration of 1-50 ms.

In any of the embodiments disclosed herein, each electric charge can deliver an energy of 0.01-150 Joules.

In any of the embodiments disclosed herein, the biological tissue can be a human heart.

In any of the embodiments disclosed herein, the human heart can be in a state of tachycardia or fibrillation.

In any of the embodiments disclosed herein, the human heart can be in a state of atrial tachycardia or fibrillation.

In any of the embodiments disclosed herein, the human heart can be in a state of ventricular tachycardia or fibrillation.

Another embodiment of the present disclosure provides a system for reducing and/or terminating arrhythmic electrical states in a biological excitable tissue. The system can comprise a plurality of electrode pairs and a controller. The plurality of electrode pairs can be configured to be placed around the biological excitable tissue. The controller can be configured to sequentially apply an electric charge to of the plurality of electrode pairs.

In any of the embodiments disclosed herein, each electric charge applied to the plurality of electrode pairs can be configured to stimulate a distinct portion of the biological excitable tissue.

In any of the embodiments disclosed herein, the plurality of electrode pairs can comprise: a first electrode pair configured to be placed around a first portion of the biological excitable tissue; and a second electrode pair configured to be placed around a second portion of the biological excitable tissue.

In any of the embodiments disclosed herein, the controller can be configured to: apply a first electric charge across the first pair of electrodes; and, after applying the first electric charge across the first pair of electrodes, apply a second electric charge across the second pair of electrodes.

In any of the embodiments disclosed herein, the controller can be configured to sequentially apply the electric charge to the plurality of electrodes to cause a rotating electric field across the biological excitable tissue.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides a flow chart illustrating a method of reducing arrhythmic electrical states in a biological tissue, in accordance with some embodiments of the present disclosure.

FIG. 2 provides a diagram of a system for reducing arrhythmic electrical states in a biological excitable tissue, in accordance with some embodiments of the present disclosure.

FIGS. 3A-D provide an image of 3D canine heart reconstructed at a 12-micron resolution using micro-CT (FIG. 3A); an image of a 2D section scan of the canine heart (FIG. 3B), experimental canine atria showing more activations by virtual electrodes following increasing strong electric shocks, in which each frame represents the same time post-shock for different strengths (FIG. 3C), and computer simulation using the atrial section from FIG. 3B showing how the pectinate muscles and vessel structures act as virtual electrodes, with more activations recruited as electric field strength increases (FIG. 3D), all in accordance with some embodiments of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As discussed above, current treatment protocols to treat arrhythmias in the heart require the application of a large shock to the heart tissue. The inventors, however, previously proposed a method of applying a series of smaller electric pulses applied to the heart tissue, which is referred to as Low Energy Anti-fibrillation Pacing (LEAP). Such a method is disclosed in U.S. Pat. No. 8,886,309, entitled “Apparatus for and method of terminating a high frequency arrhythmic electric state of a biological tissue,” which is incorporated herein by reference in its entirety as if fully set forth below. In the conventional LEAP method, however, the series of pulses/shocks are applied to a single electrode pair over a period of time. Some embodiments of the present invention improve upon this conventional LEAP method by utilizing a plurality of electrode pairs spatially dispersed around the heart, by which a series of pulses shocks can be sequentially applied to the electrode pairs, which can result in a rotating electric field applied to the heart. This can allow a larger portion of the heart muscle to be stimulated while subjecting the patient to lower energy levels.

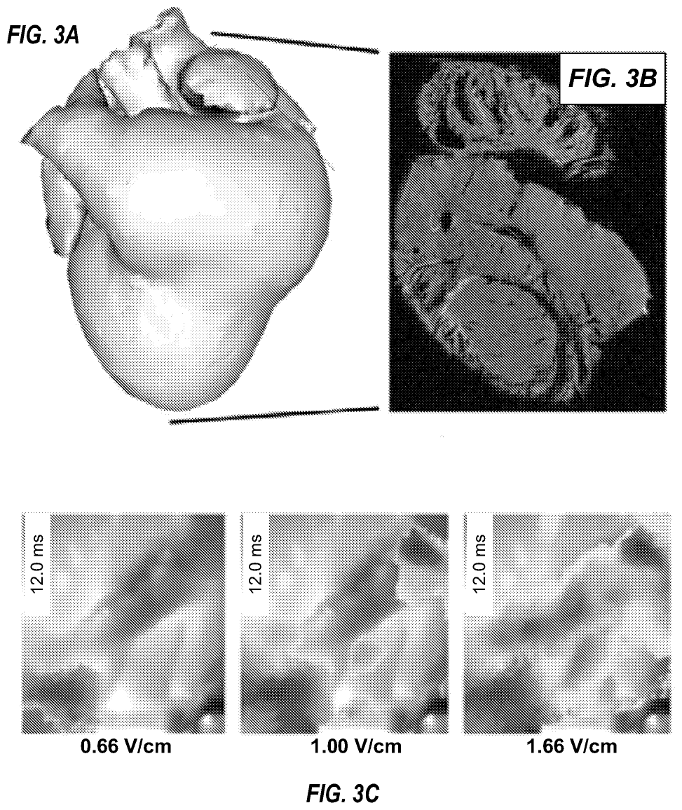

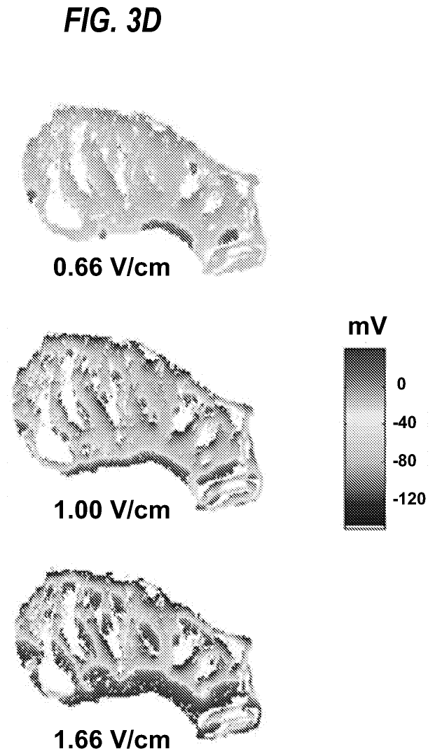

This improved LEAP method can employ low-amplitude, pulsed electric fields generated by field electrodes located within or in proximity to the atria or ventricles. The inventors have hypothesized that this technique creates many virtual electrodes—that is, regions in the tissue capable of initiating action potential waves—in the vicinity of anatomical heterogeneities located throughout the cardiac tissue (atrial and ventricular myocardium). These virtual electrodes take the place of multiple, physically implanted electrodes, which can be a more preferable clinical scenario. While based in electric fields, in some embodiments, this improved LEAP technique can use a new innovative approach of using only low energy pulses and a different mechanism to defibrillate tissue compared to the standard approach of one large shock for cardioversion/defibrillation. This innovative technique can take advantage of two known electrical characteristics of the heart. First, transthoracic defibrillation data show that during diastole, the heart can be excited by electrical field stimulation, which has 6 times smaller shock voltages (that is, 36 times less energy) than are required to defibrillate the same heart. Second, internal excitations by virtual electrodes can be produced by boundaries and heterogeneities in response to electric field shocks (See, e.g., FIGS. 3A-D). Furthermore, the time needed for exciting the whole transmural volume of a tissue, for example, the left ventricular free wall, after an external electrical far-field stimulation decreases as shock strength increases, which indicates that by increasing the electrical field shock above diastolic threshold, it is possible to recruit more and more sites of excitation (See FIG. 3C) that can defibrillate the heart. Moreover, because excitation sites can be located close to the cores of anatomical or functional reentries and to focal activity, they can facilitate entrainment of the surrounding area, even when using frequencies lower than the arrhythmia frequency.

Embodiments of the present disclosure can employ several innovative techniques. For example, activations within the tissue can be controlled. Using far-field stimulation, many virtual electrodes can be simultaneously created and centered around structural heterogeneities throughout the tissue, the number of these electrodes can be controlled by varying the field strength, and the entire tissue can be entrained using relatively low-strength fields. The defibrillation efficacy can also be improved. For example, the improved LEAP techniques can have significantly higher efficacy due, at least in part, to pacing via far-field stimulation electrodes, compared to single-site pacing from an ICD pacing electrode. This is due to the large number of virtual electrodes that can be activated. The limited range of any given electrode can be overcome by the large numbers of virtual electrodes that can be recruited with relatively low energies that can then synchronize the tissue and terminate the arrhythmia. Further, embodiments of the present disclosure can employ lower energy to defibrillate the tissue. For example, in some embodiments, the improved LEAP techniques can be at least as effective as traditional single-shock cardioversion, but with lower total energy delivered to the heart, e.g., 10-20% or less. In some embodiments, this objective can be achieved by optimizing the geometry of the pacing shock field, waveforms of the pacing shocks, and variation of the electrical field direction within and/or between the individual pacing shocks in the LEAP stimuli train as well as the time between stimuli in the train and the timing of initiation of the LEAP pulses with respect to the ECG signal.

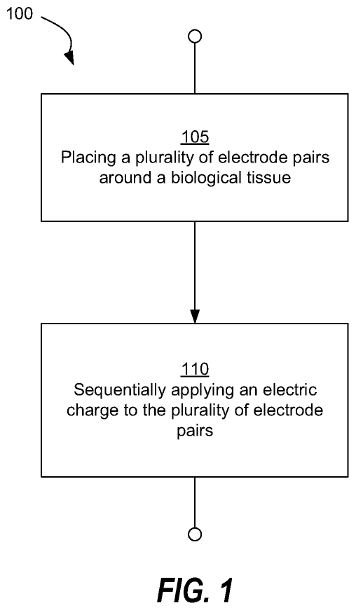

As shown in FIG. 1, an exemplary embodiment of the present disclosure provides a method of reducing arrhythmic electrical states in a biological tissue 100. The biological tissue can be many different biological excitable tissues (i.e., a tissue that can be repeatedly activated when exposed to an electric field about a threshold), including muscle tissues, nerve tissues, brain tissue and the like. In some embodiments, the biological tissue can be a human heart. In particular, embodiments of the present disclosure can provide relief to human hearts suffering from tachycardia or fibrillation—either atrial or ventricular tachycardia or fibrillation.

The method 100 can comprise placing a plurality of electrode pairs around the biological tissue 105. The electrodes can be any electrodes known in the art. In some embodiments, the electrodes can be placed around the biological tissue via surgical implant, e.g., catheter. In some embodiments, the electrodes can be used to create virtual electrodes from far field stimulations. The present disclosure is not limited to any number of electrode pairs. For example, in some embodiments, two electrode pairs are used, while in other embodiments, three, four, five, etc. electrode pairs can be used.

The electrode pairs can be placed at various positions around the biological tissue. For example, in some embodiments, the method can comprise placing a first electrode pair around a first portion of the biological tissue, and placing a second electrode pair around a second portion of the biological tissue (in some embodiments, additional electrode pairs are also utilized). The location of the electrode placement can be based on a desired stimulation result from electric pulses applied to the electrodes (discussed below). Thus, the location of the placement can be dependent on the biological tissue and the intended effect of the method (e.g., treating abnormal dynamics in the biological tissue).

The method can further comprise sequentially applying an electric charge to of the plurality of electrode pairs 110. For example, in some embodiments, the method 100 can comprise applying a first electric charge across a first pair of electrodes; and, after applying the first electric charge across the first pair of electrodes, applying a second electric charge across a second pair of electrodes. In some embodiments, each electric charge applied to the plurality of electrode pairs can stimulate a distinct portion of the biological tissue. For example, in some embodiments, a first charge applied to a first electrode pair can stimulate a first portion of the biological tissue and a second charge applied to a second electrode pair can stimulate a second portion of the biological tissue. As a person of skill in the art would understand, the first and second portions can have some amount of overlap, as the electrical fields applied to the portions can extend, at least some amount, throughout the biological tissue, but the response (activation) can be improved.

In some embodiments, sequentially applying the electric charge to each of the plurality of electrodes 110 can result in a rotating electric field (e.g., a three-dimensional rotating electric field) across the biological tissue. This can result in a greater percentage of the biological tissue being stimulated with minimal energy, as shown in FIG. 3.

In some embodiments, the electric charges applied to the plurality of electrodes can be electric charge pulses. The electric charge pulses can have varying durations and energy levels. For example, in some embodiments, each pulse can have a duration of at least 1 ms, at least 5 ms, at least 10 ms, at least 15 ms, at least 20 ms, at least 25 ms, at least 30 ms, at least 35 ms, at least 35 ms, at least 40 ms, at least 45 ms, or at least 50 ms. Additionally, in some embodiments, each pulse can have a duration of no more than 100 ms, no more than 75 ms, no more than 50 ms, no more than 45 ms, no more than 40 ms, no more than 35 ms, no more than 30 ms, no more than 25 ms, no more than 20 ms, no more than 15 ms, no more than 10 ms or no more than 5 ms. Further, each pulse can have a duration ranging between any of the foregoing values, e.g., 1-100 ms, 5-50 ms, 30-40 ms, and the like. Similarly, each pulse can deliver varying amounts of energy, in accordance with various embodiments of the present disclosure. For example, in some embodiments with internal electrodes, each pulse can deliver at least 0.01 Joules, at least 0.05 J, at least 0.1 J, at least 0.5 J, at least 1 J, at least 2 J, at least 5 J at least 10 J at least 15 J or at least 20 J. When using external electrodes each pulse can deliver at least 0.1 J, at least 0.5 J, at least 1 J, at least 2 J, at least 5 J, at least 10 J at least 20 J at least 50 J at least 100 J at least 150 J. Further, in some embodiments with internal electrodes, each pulse can deliver no more than 20 J, no more than 15 J, no more than 10 J, nor more than 5 J, no more than 2 J, or no more than 1 J . In some embodiments with external electrodes, each pulse can deliver no more than 150 J, no more than 100 J, no more than 50 J, no more than 20 J, no more than 10 J, no more than 5 J, no more than 2 J, or no more than 1 J . Further each pulse can deliver a range of energy levels between any of the foregoing values, e.g., 1-15 J if internal electrodes, 10-150 J if using external electrodes.

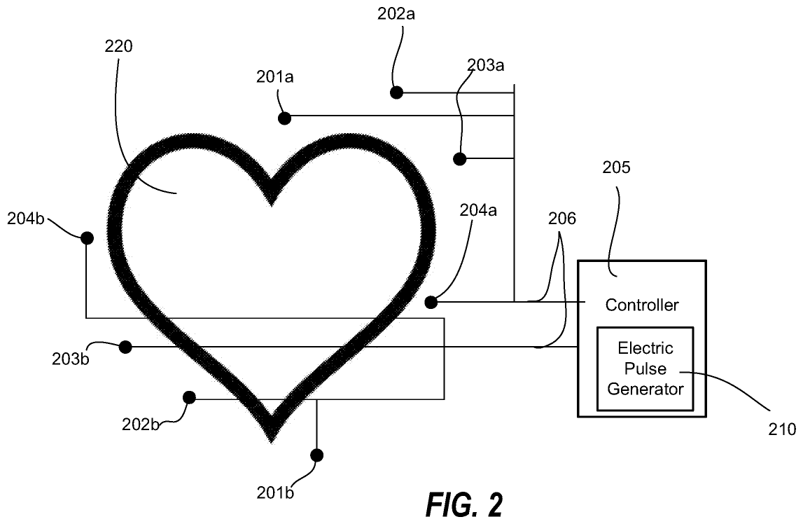

As shown in FIG. 2, another embodiment of the present disclosure provides a system for reducing arrhythmic electrical states in a biological excitable tissue. The system can comprise a plurality of electrode pairs 201a-b, 202a-b, 203a-b, 204a-b and a controller 205. Though the embodiment shown in FIG. 2 includes four pairs of electrodes, the present disclosure is not limited to any particular number of electrode pairs. The plurality of electrode pairs 201a-b, 202a-b, 203a-b, 204a-b can be configured to be placed around a biological tissue 220 as discussed above. The controller 205 can be configured to sequentially apply an electric charge to of the plurality of electrode pairs 201a-b, 202a-b, 203a-b, 204a-b as also discussed above.

The controller 210 can be many controllers known in the art, including, but not limited to a microcontroller, a computing device, and the like. In some embodiments, the controller can comprise an electric pulse generator 215. The plurality of electrode pairs 201a-b, 202a-b, 203a-b, 204a-b can be coupled to the electric pulse generator to receive electric pulses therefrom. The electric pulses can have many different waveforms, including monophasic, biphasic (which can be different shapes), and the like.

The controller can also comprise a plurality of electrical connections 206 to couple the controller 210 to the plurality of electrode pairs 201a-b, 202a-b, 203a-b, 204a-b. Accordingly, the controller, via the plurality of electrical connections 206, can apply the different electrical pulses to the various electrode pairs. For example, the controller 205 can apply a first electrical pulse having a first energy and a first duration to a first electrode pair 201a 201b, a second electrical pulse having a second energy and a second duration to a second electrode pair 202a 202b, and so on for each of the plurality of electrode pairs. As discussed above, the electrical pulses can be applied in a sequential manner to the plurality of electrode pairs 201a-b, 202a-b, 203a-b, 204a-b to create a rotating electric field around the biological tissue 220.

In some embodiments, the controller 205 can utilize machine learning techniques to determine an optimal number of electrodes, pulse energy, pulse duration, pulse frequency, and/or order of pulse application to the electrodes to best combat the arrhythmia present in the biological tissue. For example, in some embodiments machine learning techniques can be employed to determine the best time to defibrillate the biological tissue according to the dynamical state of the arrythmia experienced by the tissue. Additionally, in some embodiments, machine learning techniques can determine parameters of the pulses that allow for defibrillation by applying a minimal total energy to the biological tissue.

In some embodiments, the system can be implanted into a user having an arrhythmic condition in a biological tissue 220. Thus, in some embodiments, the system can be made of biocompatible materials and can further comprise a power source, such as a battery (not shown).

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A method of reducing arrhythmic electrical states in a biological tissue, comprising: tissue;
sequentially applying electric charge pulses with an energy of 0.01-5 Joules around the biological tissue.

2. The method of claim 1, wherein each electric charge pulse stimulates a distinct portion of the biological tissue.

3. (canceled)

4. The method of claim 1, wherein sequentially applying electric charge pulses comprises:
applying a first electric charge across a first pair of electrodes; and
after applying the first electric charge across the first pair of electrodes, applying a second electric charge across a second pair of electrodes.

5. The method of claim 1, wherein sequentially applying the electric charge pulses results in a rotating electric field across the biological tissue.

6. The method of claim 1, wherein sequentially applying the electric charge pulses results in a three-dimensional rotating electric field across the biological tissue.

7. (canceled)

8. The method of claim 1, wherein each electric charge pulse has a duration of 1-50 ms.

9. (canceled)

10. The method of claim 1, wherein the biological tissue is a human heart.

11. The method of claim 10, wherein the human heart is in a state of tachycardia or fibrillation.

12. The method of claim 11, wherein the human heart is in a state of atrial tachycardia or fibrillation.

13. The method of claim 11, wherein the human heart is in a state of ventricular tachycardia or fibrillation.

14. A system for reducing arrhythmic electrical states in a biological excitable tissue comprising:
electrode pairs configured to be placed around the biological excitable tissue; and
a controller configured to sequentially apply electric charge pulses with an energy of 0.01-5 Joules to the electrode pairs.

15. The system of claim 14, wherein the controller is further configured to apply each electric charge pulse to the electrode pairs to stimulate a distinct portion of the biological excitable tissue.

16. (canceled)

17. The system of claim 15, wherein the controller is further configured to:
apply a first electric charge across a first pair of electrodes; and
after applying the first electric charge across the first pair of electrodes, apply a second electric charge across a second pair of electrodes.

18. The system of claim 14, wherein the controller is further configured to sequentially apply the electric charge pulses to the electrodes to cause a rotating electric field across the biological excitable tissue.

19. (canceled)

20. The system of claim 18, wherein each electric charge pulse has a duration of 1-50 ms.

21. (canceled)

22. The system of claim 14, wherein the biological tissue is a human heart.

23. The system of claim 22, wherein the human heart is in a state of tachycardia or fibrillation.

24. The system of claim 23, wherein the human heart is in a state of atrial tachycardia or fibrillation.

25. The system of claim 23, wherein the human heart is in a state of ventricular tachycardia fibrillation.
